(19) United States
(12) Patent Application Publication
(10) Pub. No.: US 2011/0135754 A1
(43) Pub. Date: Jun. 9, 2011

(54) CONTROL OF DISEASES IN CROPS

(76) Inventors: Fred Chresten Hoberg, New South Wales (AU); Jeanette Lillian Hoberg, New South Wales (AU)

(21) Appl. No.: 13/058,077

(22) PCT Filed: Aug. 3, 2009
(86) PCT No.: PCT/AU2009/000993

§ 371 Date: Feb. 8, 2011

(30) Foreign Application Priority Data
Aug. 8, 2008 (AU) 2008904060

(51) Int. Cl. A61K 33/24; C01G 31/02; A61P 31/10

(52) U.S. Cl. 424/646; 423/594.17

(57) ABSTRACT

A composition and a method for controlling a fungal disease, such as rust, in a crop, such as a grain crop, where said composition contains a vanadium containing compound that can be applied to the seed, or to the crop, or to the ground before, during or after seeding.

### TECHNICAL FIELD

The current invention relates to a method and a product for controlling diseases, such as fungal diseases, that affect crops, in particular those crops that have developed from grasses such as wheat crops and the like.

### BACKGROUND ART

Fungal diseases and infestations affect many crops around the world. One fungal infestation, commonly known as “rust” affects wheat production in most areas of Australia to some degree. The rust infestation has been a problem in Eastern Europe for many years, and there have been recent reports of this infestation crossing the Red Sea into Asia, where it had not hitherto been a problem. The spread of this infestation on food crops, combined with the general food shortages world wide due to climate changes and the increasing demand for bio-fuels, has placed greater urgency on being able to control the debilitating affect of fungal diseases on crops.

There are currently numerous man made biocides for controlling fungal infestations that affect crops. Halogen containing hydrocarbon based compounds are one such type of biocide. These compounds have been used on crops for over 50 years. However, these compounds are relatively expensive, create potential unwanted environmental side effects, and in some cases may possibly have lead to pesticide resistant species evolving.

It is thus desirable to have a more economical and simple method for treating fungal diseases in crops.

### SUMMARY OF INVENTION

The current invention relates to adding vanadium or a vanadium containing compound to a crop environment to control, including minimizing, or preventing, or treating fungal diseases such as rust.

Most soils contain living organic material (i.e. biomass) that can produce halogen containing hydrocarbons through the action of soil microbes. Certain halogen containing hydrocarbons are known to be effective in treating or controlling many fungal diseases associated with crops such as rust. There is much literature relating to the biological production of halogenated hydrocarbons including those of both aquatic and terrestrial origin. Volcanic and other tectonic activity also contribute enormous amount of halogens as elemental material or simple inorganic compounds. Through rainfall and volcanic activity it is expected that halogenated compounds would be found on most of the earth's surface, especially those areas deemed suitable for agriculture.

It is further known that elemental vanadium may be involved as a catalyst on the soil microbes in the biomass to assist in the production of halogen containing hydrocarbons. However the inventor has discovered that most soils may be lacking in having vanadium compounds that can act as a catalyst for the soil microbes. In addition, vanadium may also catalyse soil microbial nitrogenase enzyme systems. To date, there are no methods in use that use vanadium for the purpose of acting as a catalyst for the soil microbes to produce halogen containing hydrocarbons to control or treat fungal diseases in crops.

Therefore in one aspect of the invention there is provided a composition for controlling a fungal disease in a crop, where said composition contains vanadium or a vanadium containing compound.

In one embodiment of the invention, the fungal disease being treated or controlled is rust.

In another embodiment of the invention, the crop is one that has been developed from grasses. This may include staple grain crops such as wheat, barley, oats or rye, or stock feed crops such as tritacle.

In another embodiment of the invention the vanadium is present in the composition in a soluble form and may include for example, either the vanadate or vanadyl form.

In another embodiment of the invention the vanadium is present in the composition in a solid form, preferably a finely, evenly dispersible solid form.

Preferably, the composition is a solution that can be sprayed, onto a growing crop, or onto the ground before, during, or after seeding.

In a further embodiment of the invention the vanadium or vanadium containing compound can be applied directly to the crop seed at planting or mixed with the crop seed, prior to planting.

Preferably the composition when in a solution form, would be of a concentration to apply 5-25 grams of elemental vanadium per hectare. The concentration of vanadium in the solution would be dependent upon the rate of application. It is also understood that the concentration required may vary depending upon many variables, such as the precise fungal disease to be controlled, the type of crop, and the amount of vanadium that may already be present in the soil.

The composition may additionally contain halogen containing compounds. In particular, a bromine salt may also be included in the composition.

In another aspect of the invention there is provided a method for controlling a fungal disease in a crop, where said method includes applying to either a growing crop or the ground where a crop has been planted or about to be planted, or the crop seed prior to planting, a composition vanadium or a vanadium containing compound.

### DETAILED DESCRIPTION OF THE INVENTION

It is considered, that at least in Australia, the majority of soils have a sufficient biomass and soil microbes to potentially produce sufficient halogen containing hydrocarbons that can control fungal diseases such as rust, if vanadium were present to act as a catalyst. However as soil compositions do vary dramatically, tests can be done on the soils to see what deficiencies the soil may contain. For example, if it is found that the soil biomass is lacking in halogen content, to be able to produce the desired amount of halogen containing hydrocarbon compounds, a halogen, such as bromine in the form of sodium bromide, may be further added to the composition. Alternatively or additionally, if the soil is found to be deficient in having the requisite soil microbes to produce the required halogen containing hydrocarbons to control fungal diseases, the appropriate microbe culture could be added. However, this is expected to only be required in rare circumstances, as it is expected that most soils that are to be cropped for grain or stock feed would be sufficiently fertile and contain sufficient amounts of microbes to produce halogen-containing hydrocarbons.

Any soluble compound of vanadium may be used. Compounds of the vanadyl complex (VO++) are suitable because they are simple to prepare from the most economical and available form of vanadium (vanadium pentoxide, V2O5), are stable, and mostly very soluble. Vanadyl salts of citric acid, succinic acid, glutaric acid, adipic acid, and ethylene diamine tetra acetic acid may also be suitable.

In the vanadyl form, vanadium is in a reduced state (oxidation state 4) and on exposure to the atmosphere it can be oxidised to the higher oxidation state 5 such as vanadate and vanadium pentoxide. The latter is only slightly water soluble and if present in water at above the saturated state it forms a solid precipitate and is therefore not as readily dispersible. However the oxidation of the vanadyl form appears to be slow enough to allow adequate dispersion within the soil micro environment. Vanadate forms such as ammonium meta vanadate are already in the highest oxidation state (5) and are therefore resistant to further oxidation. Vanadium Pentoxide is sparingly soluble (0.8 g/100 ml at 20 c) and could be dispersed in solution. In bulk, vanadyl salts oxidise very slowly and can be protected from oxidation by addition of a reducing agent such as sodium meta bisulphite.

At an application rate of 5 g of elemental vanadium per hectare (1000 m2) the resulting concentration of elemental vanadium in soil when distributed evenly to a depth of 0.075 m would be approximately 5.5 parts per billion (ppb) by weight. While 5.5 ppb would appear to be an extremely low concentration by the usual standards for soil mineral nutrition the presence of vanadium in the soluble form may account for efficacy above the level expected of that low concentration.

Typically terrestrial soils have vanadium concentration in the range of 10 to 100 ppm of which only a small proportion would be soluble in most circumstances.

An embodiment of the invention will now be described in the following example.

### EXAMPLE 1

A solution containing vanadium in the form of vanadyl citrate was prepared. The solution was sprayed onto about 2 hectares of an oats crop for stock feed, that was in early stages of growth where the crop was approximately 150 mm in height. The spray was applied in a manner such that approximately 7.5 grams of elemental vanadium per hectare, was applied to the crop.

The crop that was treated with the solution, delivered three cuttings, whereas an adjacent crop that was left untreated, failed to give any yield due to rust.

The claims defining the invention are as follows:

1. A composition for controlling a fungal disease in a crop, where said composition contains a vanadium containing compound.

2. The composition of claim 1, where the fungal disease being controlled is rust.

3. The composition of claim 1, where the crop is of the type that has been developed from grasses.

4. The composition of claim 1, where the crop is a grain crop.

5. The composition of claim 4, where the grain crop is wheat.

6. The composition of claim 1, where the vanadium is present in the composition in a soluble compounded form.

7. The composition of claim 1, where the vanadium is present in a solid compounded form.

8. The composition of claim 1, where the composition is a solution that can be sprayed onto a growing crop, or onto the ground before, during, or after seeding.

9. The composition of claim 8, where the composition when in solution form, would be of a concentration such that 5-25 grams of vanadium can be applied per hectare.

10. The composition of claim 1 where the composition is applied to a crop seed prior to planting.

11. The composition of claim 1, where the composition may additionally contain a halogen containing compounds.

12. The composition of claim 11, where the halogen containing compound is a bromine salt.

13. A method for controlling a fungal disease in a crop, where said method includes applying to either a growing crop or the ground where a crop has been planted or about to be planted, or the crop seed prior to planting, a composition containing a vanadium compound.

14. The method of claim 13, where said fungal disease is rust.

15. The method of claim 13, where said crop is of the type that has developed from grasses.

16. The method of claim 15, where said crop is a grain crop.

17. The method of claim 16 where said grain crop is a wheat crop.

18. The method of claim 13, where the vanadium is present in a soluble form and applied to the crop or the seed, or the ground in solution form via a spray method.

19. The method of claim 18, where 5-25 grams of elemental vanadium is applied per hectare.

20. The method of claim 13, where a halogen containing compound is also applied.

21. The method of claim 20, where the halogen containing compound is a bromine containing compound.
